(19) United States
(12) Patent Application Publication
(10) Pub. No.: US 2026/0083932 A1
(43) Pub. Date: Mar. 26, 2026

(54) ODOR-BASED TMR SYSTEM AND METHOD

(71) Applicant: Justin Dumitrescu, Toronto (CA)

(72) Inventor: Justin Dumitrescu, Toronto (CA)

(21) Appl. No.: 19/333,907

(22) Filed: Sep. 19, 2025

Related U.S. Application Data
(60) Provisional application No. 63/699,303, filed on Sep. 26, 2024.

(51) Int. Cl. A61M 21/00

(52) U.S. Cl. CPC A61M 21/00; A61M 2021/0016

(57) ABSTRACT

An Odor-based TMR System for enhanced learning and memory is disclosed herein. The TMR system and method comprises a fragrance and a physical delivery system.

### CROSS-REFERENCE TO RELATED APPLICATIONS

The present application claims priority to U.S. provisional application Ser. No. 63/699,303 filed Sep. 26, 2024, herein incorporated by reference in its entirety.

### BACKGROUND

The invention generally relates to Targeted Memory Recall (“TMR”) and the need for an easily accessible, affordable and efficacious method of harnessing its potential.

Memory consolidation during sleep has been a focus of research for decades, with early studies revealing the critical role of sleep in stabilizing and enhancing learned information. Targeted Memory Reactivation (TMR) builds on this foundation by demonstrating that sensory cues associated with prior learning, when presented during sleep, can selectively strengthen specific memories. Initial experiments by Rasch et al. (2007) and others revealed that auditory and olfactory cues could successfully reactivate and enhance memory traces, providing compelling evidence for the role of sensory reactivation in sleep-dependent memory consolidation.

Notably, Neumann et al. further expanded on this work by showing that rose odor presented during learning, sleep, and retrieval phases improved memory performance in a real-world setting. This finding highlights the potential of olfactory cues as effective tools for TMR, particularly due to their robust association with memory and ease of delivery.

This approach aims to enhance memory consolidation in a way that is both effective and practical. By leveraging a straightforward delivery method, this research seeks to bridge the gap between laboratory findings and real-world application. The results have the potential to increase the accessibility and scalability of TMR interventions, paving the way for practical applications in educational and therapeutic contexts while contributing to a deeper understanding of sleep-related memory processes.

The majority of TMR efforts are audio cue based and/or targeted towards wearables with interfaces to smart-phones or other devices. There are downsides to these concepts including cost, adoptability and efficacy. In terms of costs, wearables are expensive to create and manufacture and are typically purchased only once which leads to the need for a high retail price.

Low consumer adoption. Ensure a reasonable retail cost and avoid systems that may be uncomfortable and/or disrupt sleep (such as wearables). As mentioned above, there are downsides to the concepts currently being investigated/developed including required retail cost, consumer adoptability and efficacy.

In terms of efficacy, there is research showing that auditory cues are inferior to olfactory cues in terms of memory reactivation. Also, anything that disrupts a person's sleep will lead to lower efficacy with TMR. This disruption can result from audio cues themselves or the actual wearable.

The present invention attempts to solve these problems, as well as others.

### SUMMARY OF THE INVENTION

Provided herein are systems and methods for TMR.

The systems and methods are set forth in part in the description which follows, and in part will be obvious from the description, or can be learned by practice of the systems and methods. The advantages of the systems and methods will be realized and attained by means of the elements and combinations particularly pointed out in the appended claims. It is to be understood that both the foregoing general description and the following detailed description are exemplary and explanatory only and are not restrictive of the systems and methods, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g. ” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF EMBODIMENTS

Generally speaking, the TMR system and method comprises a physical delivery system including proprietary fragrances, and the protocol for using the delivery system to achieve enhanced memory recall for a subject. Primary intended uses would be learning or retaining information and/or attaining or refining a motor skill, including but not limited to, students, athletes and employees. The method comprises applying the fragrances to a subject during a learning phase (e.g. at least one hour during learning) and during a sleep phase; and repeating as often as desired to achieve enhanced memory recall.

The delivery system comprises an applicator product including dosed nasal strips, lip balm or similar lip products, roll-on applicators, cosmetic applicators, oils, lotions, or other skincare products. The delivery system comprises proprietary fragrances including roses or mint. Proprietary fragrances are selected from effective TMR scents that are less likely to already be heavily tied to existing memories. For example, the smell of cinnamon is likely attached to a variety of memories and would be less efficacious at generating TMR. Conversely, a custom scent comprising Hinoki wood, Blue tansy and Ambergis is unlikely to be attached to previous memories as it is unlikely a consumer has encountered this scent before. Proprietary fragrances include custom scents that are designed to last for between about three hours to about four hours. TMR is most efficacious when curing memories during slow wave sleep. The majority of slow wave sleep occurs during the first four hours of sleep.

Consumers interested in learning new subject matter or attaining a skill use the product during study, practice, acquiring a skill, or learning some subject matter and then again before sleep. TMR has been proven to work after one night but TMR effects are compounded over multiple days/nights. Depending on the delivery system, the product would last for either one week (nose strips) of multiple weeks (lip or roll on product).

This odor-based TMR system is significantly less expensive as it does not require expensive technology and hardware.

There are no TMR products currently on the market, the ones being developed are focused primarily on audio-based cues or are heavily tech focused relying on audio or odor hardware delivery systems combined with wearables that can indicate your sleep state. Delivering sleep cues only during a specific sleep state is known as Brain State Dependent TMR whereas delivering cues throughout the entire night is known as Brain State Independent TMR. The TMR system is significantly less expensive as it does not require expensive technology and hardware (both delivery system and sleep state measuring system). In terms of efficacy, there is research showing that audio is inferior to odor in terms of memory cueing. Also, anything that can disrupt a person's sleep will lead to poor results with TMR. Disruption can result from audio cues themselves or the actual wearable.

### OTHER EMBODIMENTS

Emotional Regulation: In one embodiment, the TMR system and method is used to reinforce positive emotional responses or reduce negative ones by reactivating memories or experiences associated with specific emotions. This TMR system and method would beneficial in therapeutic settings, particularly for conditions like anxiety or depression.

Habit Formation: In one embodiment, the TMR system and method is used to strengthen the neural pathways associated with the formation of new habits or the breaking of old ones. By targeting and reinforcing desired behavioral patterns, TMR system and method helps in habit training.

Treatment of PTSD: In a controlled manner, the TMR system and method is used to modify the emotional responses tied to traumatic memories, aiding in the treatment of PTSD by gradually desensitizing individuals to their triggers. The TMR system and method is used by individuals with disorders for which memory disruption is desired such as post-traumatic stress disorder (PTSD), obsessive compulsive disorder, depression, or other disorders appreciated by one skilled in the art.

Creativity Enhancement: By reactivating specific memories or thought patterns, the TMR system and method is be used to foster creative thinking, helping to make connections between seemingly unrelated ideas or concepts during sleep.

Cognitive Behavioral Therapy (CBT) Enhancement: TMR system and method supports CBT by reinforcing positive cognitive patterns or behaviors learned during therapy sessions, potentially speeding up the therapy process.

Addiction Treatment: TMR system and method is used to weaken the neural connections associated with addictive behaviors or cravings, supporting efforts to quit addictive substances or behaviors.

In one embodiment, the TMR system and method is used to improve memory and/or cognitive function by individuals with cognitive and/or memory deficits associated with normal aging or neurodegenerative disorders including Alzheimer's disease, Parkinson's disease, frontotemporal dementia, and other age-related or neurodegenerative disorders appreciated by one skilled in the art.

In one embodiment, the TMR system and method is used as part of a therapeutic method to treat a patient. For example, the devices, systems and methods is used to improve memory and/or cognitive function by individuals with inherited neurodevelopmental disorders characterized by learning, memory and/or cognitive deficits including Down syndrome, Rett syndrome, fragile X syndrome, neurofibromatosis type 1, tuberous sclerosis, phenylketonuria, maple syrup urine disease, and other inherited neurodevelopmental disorders appreciated by one skilled in the art, as well as disorders such as autism spectrum disorders which are generally diagnosed in the first five years of life and may be due to genetic and/or environmental causes.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example: Physical Delivery System

The TMR system and includes physical delivery systems selected from cosmetic products such as chapstick, roll-on applicators or nasal strips. The TMR system is not sleep dependent TMR and therefore does not require expensive tech or hardware to identify sleep states. Instead, the TMR system comprises using proprietary fragrances that would last for the first three to four hours of sleep, which covers the majority of slow wave sleep but is not brain state dependent TMR.

In one embodiment, the TMR system comprises a simple applicator product such a nasal strips or lip balm is easy to use and is not uncomfortable. This increases usability in general but particularly for use cases such as athletes attempting to learn new motor skills.

Efficacy. As mentioned, odor-based cues are superior to other cues including audio. Also, the TMR system results in a low probability of sleep disturbances as scent does not typically lead to awakenings and there is nothing uncomfortable to wear during sleep. Both of these factors lead to higher TMR efficacy.

### Example: Testing Efficacy of Olfactory Targeted Memory Reactivation

Targeted Memory Reactivation (TMR) is a technique that enhances long-term retention of memories by using sensory cues to prompt hippocampal-cortical replay of specific memories during sleep. The proposed study will test whether scented adhesive strips applied to the nose (during learning and sleep) can be used to tag and reactivate memories in the same manner previously demonstrated for other studies. The results from this study may enable us to develop new consumer products for memory enhancement.

Research Question: Can memories be selectively strengthened during sleep using olfactory targeted memory reactivation (oTMR) delivered via adhesive strips?

Hypothesis: Forgetting after 1 day and 1 week (as measured using a spatial memory task) will be reduced when people receive reactivation of learned information using oTMR compared to when they do not receive oTMR.

Objectives: This study is intended to test whether a scented nasal adhesive strip can effectively improve memory using oTMR.

Outcomes: The results from this study will help researchers develop techniques and products for using Targeted Memory Reactivation to improve memory in every life situations. Specifically, the experiment will test whether an approach that is technically simpler than previous approaches for oTMR can produce equivalent memory benefits.

This study investigates the use of adhesive nasal strips infused with distinctive scents as a novel, practical, and user-friendly method for olfactory TMR. Unlike prior methods requiring controlled laboratory setups or specialized equipment, these nasal strips offer a consistent and non-intrusive delivery system. By reactivating memory traces through familiar scents during both learning and sleep, the strips simplify the TMR process, making it accessible for broader applications.

### Study Interventions

The study involves adhesive strips applied to the bridge of the nose and outer surface of the nostrils with a weak adhesive. These strips are mechanically identical to products on the market (E.g. breath-rite strips) but release a distinctive and pleasant scent such as roses or mint.

Each participant receives 4 strips with different scents. Strips are given to the participant to self-apply during the learning phase of the experiment, and one sealed strip is also sent home with the participant following each of the learning sessions to be used at night.

Device handling: Prior to use, strips will be stored securely by the investigator. We will verify that the strips are used correctly by (1) observing the participant applying them in the lab setting and (2) asking participants to take an image of themselves with the strip applied before they sleep).

### Protocol

The experiment is a within-subjects TMR study with 48 participants who perform a spatial memory task similar to that of the original Rudoy TMR experiment and the Whitmore automated TMR study. Participants will visit the lab to perform a learning task where they learn the locations of objects on a spatial grid.

Each participant performs two experimental blocks. In each block, the participant visits the lab and performs a computerized spatial learning task in which they learn the locations of 30 objects on a grid background. Participants first view each object and practice placing it on the grid. They are provided with feedback if they place it in the correct location. After they have viewed and placed all 30 objects, they complete a “criterion test”, where objects are presented in the center of the grid and they are asked to place each object at its correct location. Participants receive feedback after placing each object, with the correct location shown. If the participant placed the center of the object within 5% of its correct location, the object is considered “learned” and is dropped from the rotation. The objects that have yet to be correctly placed are cycled through in random order until all are successfully learned. After completion of learning, participants rest for 5 minutes and then complete an immediate post-test, where each object is presented in the center of the screen and the participant is asked to move it to the correct location. No feedback is provided during post-test, but their immediate memory accuracy is measured. We anticipate that this process will take about an hour.

During the learning, participants wear an adhesive strip under their nose containing a distinctive scent. The strip is placed at the beginning of the learning and removed after completing the final test.

At the end of the block, participants are given an additional strip to wear overnight. The participant is instructed to apply the strip before going to bed and remove it when waking up in the morning. Participants are asked to take and submit a selfie each night of them wearing the strip using a secure web portal.

Following their first night of sleep, participants are emailed a link to complete a series of questionnaires on a secure web portal (Qualtrics). These consists of a brief questionnaire (Leeds Sleep Evaluation questionnaire) asking about their sleep quality the previous night, as well as their impressions of the comfort and ease of use of the scent strips. They then complete a memory test identical to the immediate post-test, where they are asked to place each object in its correct location, without receiving feedback.

Participants return to the lab for their second learning session, where they receive a different nasal strip with a different smell and complete the spatial learning task with a new set of objects. Following the visit, each participant is issued another strip to wear overnight and repeats the overnight procedure.

After the participant completes the questionnaires following the second night of sleep, they have completed the experiment and enter their email to receive a gift card for payment.

### Experimental Conditions

Each participant performs one block with “true” TMR, and one block with “Sham” TMR. The difference between these conditions is that for true TMR, the same scent is used during both learning and sleep, triggering reactivation of the learned memories during sleep. In the sham TMR condition, different scents are used for learning and sleep, so the nose strip applied during sleep does not reactivate the memories of learning. Participants are assigned to have their true block first or sham block first in alternating order.

Devices: The nose strips worn are custom-manufactured scented nose strips which are attached to the nose with a weak adhesive on the device's sticky side. These devices are not subject to regulatory approval and are custom made for the study.

The primary risks of this study are discomfort or skin reaction to the nose strips in the lab portion or while sleeping.

To mitigate these risks, we will emphasize to participants that they can reposition the nose strips (as well as withdraw from the experiment at any time during the experiment orientation. We will also encourage the participants to report any discomfort. If we or the participants notice signs of a skin reaction (rash, pain, itching, etc.), we will withdraw participants from the experiment. Participants will also be encouraged to reposition (or remove, if necessary) the nose strips they use at home if discomfort occurs.

In addition, participant data will be stored securely and anonymized to protect participant privacy, and we will delete all photos within 48 hours after they are acquired.

Photography: After applying the nose strips at night, we will ask participants to take a picture of their face with the strip on and submit it through a secure web form using a mobile device or laptop. This step is performed so we can verify that participants have correctly applied the strip at home. Photos will be securely stored in a Qualtrics instance accessible only to the study team. Photos will be inspected within 48 hours of acquisition and then deleted; we will retain only a record of whether the device was applied correctly and not the original photo.

### Data

Data will be collected using a Qualtrics instance and collected data will be accessible only to the study team. Participants will do the memory tests and questionnaires on both a lab computer and on their personal devices; the data will then be encrypted for transmission and sent to the Qualtrics instance.

All data collected in the study will be unlinked from identifiers: when participants sign up for the study, they are given a randomly generated participant code which is used to identify them. While we do collect participants emails (to send them questionnaires and payments) the emails are never linked to the participant ID in Qualtrics.

Data will be stored long-term and analyzed on the research team's local and cloud storage systems, which are also access-restricted to allow only research team members to access them. We plan to retain de-identified data indefinitely (but for at least 5 years after study completion).

The de-identified data may be shared in public data repositories, such as the Open Science Framework or publisher's repositories. It may also be shared with investors or corporate entities working with us. Transmission of data to other entities will be handled by the PI (Nathan Whitmore).

### Timelines

Each participant visits the lab twice, and each visit takes about an hour. In addition, the day after each visit, participants will complete an online memory test which will take about 15 minutes. We anticipate it will take about 4 months to enroll all study participants, and about 1 month to complete analysis after all of the participants have completed the study.

### Inclusion/Exclusion Criteria

We will include English-speaking adults ages 18-50 who are able to give informed consent do not report any current sleep disorder, memory disorder, or issues with taste/smell perception. We will exclude participants who report any allergies in the components of the adhesive band or chronic skin conditions such as dermatitis. Participants will be screened online before allowing them to schedule an appointment using the questions in the participant screening form (attached).

Participants will be included in the final analysis if they successfully complete all of the study procedures (2 lab visits and 2 online questionnaires), upload photos of themselves correctly wearing the nose strips at night, and do not self-report any major issues judged by us to compromise the data (e.g. a technical issue that prevents them from completing the home surveys accurately).

Special populations:

Adults unable to consent: Not eligible

Individuals who are not yet adults: Not eligible

Pregnant people: Eligible, but not specifically recruited

Prisoners: Not eligible

Marginalized/Minority/Vulnerable Populations: Eligible, but not specifically recruited

### Data Plan

Our primary endpoint is the amount of forgetting that occurs from the immediate test to the delayed memory test in each block. We predict that forgetting will be reduced when participants receive the same scent during learning and during sleep, compared to when they receive different scents during learning and sleep. Forgetting is quantified by (1) Obtaining the mean of placement errors (distance between an object's true location and the location the participant placed it) over all 30 objects on each test to yield one test-wide error score; (2) Subtracting the immediate test score from the delayed test score to obtain the amount that error increased over time (quantifying forgetting).

We will use a two-tailed repeated-measures test (paired T test or Wilcoxon signed-rank test if the t test requirements are not met) to test whether condition has a significant effect on forgetting (p<0.05).

We will also compare forgetting between the true TMR block and the sham TMR block using a linear mixed-effects model with the amount of forgetting as a dependent variable, subject ID as a random factor, and score on the immediate test and condition (true or sham TMR). We will test whether the factor condition significantly (p<0.05) predicts the amount of forgetting in order to verify that the observed effects cannot be explained by differences in initial test performance or block order.

We will also use a linear model to evaluate whether the difference in memory performance between the two blocks is associated with:

1. LSEQ total score

2. LSEQ quality of sleep score

3. Performance on the immediate recall test

4. Self-rated comfort of the nasal strips

5. Age

Each variable will be evaluated separately. We will also collect data on the usability of the nasal strips at the end of the experiment, using the questions in the “system usability” document—these are fore descriptive statistics only and we will not run any tests on them.

Data security: Data will be collected using a Qualtrics instance and collected data will be accessible only to the study team. Participants will do the memory tests and questionnaires on both a lab computer and on their personal devices; the data will then be encrypted for transmission and sent to the Qualtrics instance.

All data collected in the study will be unlinked from identifiers: when participants sign up for the study they are given a randomly generated participant code which is used to identify them. While we do collect participants emails (to send them questionnaires and payments) the emails are never linked to the participant ID in Qualtrics.

Data will be stored long-term and analyzed on the research team's local and cloud storage systems, which are also access-restricted to allow only research team members to access them. We plan to retain de-identified data indefinitely (but for at least 5 years after study completion).

The de-identified data may be shared in public data repositories, such as the Open Science Framework or publisher's repositories. It may also be shared with investors or corporate entities working with us. Transmission of data to other entities will be handled by the PI (Nathan Whitmore).

Quality control: For each participant, we will verify that they complete all of the required sessions (two in lab sessions and two sets of home questionnaires) and visually verify that they have attached the nose strips correctly at home using the photographs taken by participants. If participants pass these checks, their data will be included in the analysis.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. An Odor-based TMR System comprising: a delivery system and a fragrance to be applied to a subject during a learning event and applying the fragrance to the subject during a sleep event.

2. The system of claim 1, wherein the delivery system is selected from the group consisting of dosed nasal strips, lip balm, lip products, roll-on applicators, cosmetic applicators, oils, lotions, and other skincare products.

3. The system of claim 2, wherein the fragrance is selected from the group consisting of cinnamon, mint, Hinoki wood, Blue tansy and Ambergis.

4. The system of claim 3, wherein the fragrance lasts between about three hours to about four hours on the subject.

5. The system of claim 4, wherein the fragrance is applied during slow wave sleep.

6. The system of claim 5, further comprising improving memory and recall in the subject.
